(19) United States
(12) Patent Application Publication
(10) Pub. No.: US 2005/0142508 A1
(43) Pub. Date: Jun. 30, 2005

(54) Automatic power-off control circuit for essential oil burner

(76) Inventors: Chau-Young Lee, Jhonghe City (TW); Hsu-Chang Chen, Jhonghe City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/747,362

(22) Filed: Dec. 30, 2003

(51) Int. Cl. A61L 9/03

(52) U.S. Cl. 431/18; 422/125

(57) ABSTRACT

An automatic power-off control circuit of essential oil burner is provided for controlling the heating of the essential oil in the essential oil burner, and cutting off power when the essential oil in the essential oil burner is used up. The control circuit includes a power-on switch, an AC/DC transformer, a signal detection circuit, a signal amplifier, and a heating device. The power-on switch is connected in series between an AC power supply and the AC/DC transformer. The power-on switch is connected in parallel with a power self-holding circuit so that the AC power supply keeps supplying power to the heating device to heat the essential oil in the bowl when the switch is pressed and the signal detection circuit detects the presence of essential oil in the bowl of the essential oil burner. When the signal detection circuit detects that essential oil is used up in the bowl, the self-holding circuit relieves its self-holding state and cuts off the supplying of the power to the heating device. The control circuit further includes a heating indicator and a night light circuit.

### BACKGROUND OF THE INVENTION

1. Field of the Invention

This invention relates to a control circuit for essential oil burner and, more particularly, to an automatic power-off control circuit for essential oil burner.

2. Description of the Prior Art

As more and more people are concerned with improving the quality of life in the recent years, many novel equipments and facilities are manufactured to meet the growing need for those seeking quality lifestyle. The essential oil burner is one of the items that have gained much popularity due to its ease of use and reputable effects.

Conventional essential oil burners provide different designs to meet different purposes. For example, Taiwan patent publication No. 437,917 disclosed a design of essential oil burner whose burning base is placed on a container with a heating wick in the middle. The other end of the heating wick is soaked in the essential oil in the container. The burning base can be covered with an outer cap. The top rink of the burning base includes a net cover, which is padded with a specially treated platinized asbestos layer. The layer is directly in contact with the heating wick, and is air-permeable. Taiwan patent publication No. 474,182 disclosed a new structure of an essential oil burner, including a main body, which is single-mold of a cap base and a container. The cap base is a hollow case, and inside the cap base is a heating device. The upper part of the cap base is to support the open container, which is for containing water and essential oil. An AC power source is rectified and voltage lowered to generate a DC voltage, which is passed through a regulator for filtering and regulation. The final DC current is to supply the microprocessor and the impedance detection components of the heating device. The microprocessor is preset with a range of impedance resulted from contacting water. The impedance detection component detects the impedance with or without contacting water, which determines whether the microprocessor issues control signal to activate or deactivate the heating device. The impedance detection component is embedded in the bottom of the container wall of the main body in order to detect the presence of the water in the container for activating or deactivating the heating device.

### SUMMARY OF THE INVENTION

In the conventional essential oil burner design, the safety and the convenience of the circuit is not well considered. For example, it is a safety hazard if the user forgets to turns off the power when the essential oil in the essential oil burner is used up.

Because of the shortcomings in the conventional designs, the first object of the present invention is to provide an essential oil burner with an automatic power-off control circuit. With the automatic power-off control circuit, the safety and the convenience of using essential oil burners can be improved.

Another object of the present invention is to provide an automatic power-off control circuit for an essential oil burner. When the users use an essential oil burner, they only need to press a simple power-on switch to activate the essential oil burner. When the essential oil is used up, the control circuit can automatic detects its state and cut off the power supply.

Yet another object of the present invention is to provide an automatic power-off control circuit with a self-holding circuit for an essential oil burner. When the users press the power-on switch to activate the essential oil burner, the control circuit can keep supplying power to the heating device of the essential oil burner in a self-holding manner. This improves the operational simplicity.

To solve the aforementioned problems and achieve the objects, the present invention of a control circuit includes a power-on switch, an AC/DC transformer, a signal detection circuit, a signal amplifier, and a heating device. The power-on switch is connected in series between an AC power source and the AC/DC transformer. The power-on switch is connected in parallel with a power self-holding circuit so that the AC power supply keeps supplying power to the heating device to heat the essential oil in the bowl when the switch is pressed and the signal detection circuit detects the presence of essential oil in the bowl of the essential oil burner. When the signal detection circuit detects that essential oil is used up in the bowl, the self-holding circuit relieves its self-holding state and cuts off the supplying of the power to the heating device.

For the preferred embodiment of the present invention, the control circuit can further include a heating indicator to indicate the heating state of the heating device and a night light circuit so that the essential oil burner can also serve as a night light.

Through the technologies adopted in the present invention, the essential oil burner can detect that the essential oil in the burner is used up and automatically cut off the power when it is in use. This greatly improves the safety of essential oil burner usage. Furthermore, the self-holding circuit of the control circuit of the present invention allows the user only need to press the power-on switch to activate the essential oil burner. The self-holding feature of the control circuit can keep the power supplied to the heating device. This improves the operational convenience.

These and other objects, features and advantages of the invention will be apparent to those skilled in the art, from a reading of the following brief description of the drawings, the detailed description of the preferred embodiment, and the appended claims.

### BRIEF DESCRIPTION OF THE DRAWINGS

FIG. 1 shows a schematic side of an embodiment of the present invention;

FIG. 2 shows a block diagram of the control circuit of the present invention; and

FIG. 3 shows a detailed control circuit diagram of the present invention.

### DETAILED DESCRIPTION OF THE PREFERRED EMBODIMENTS

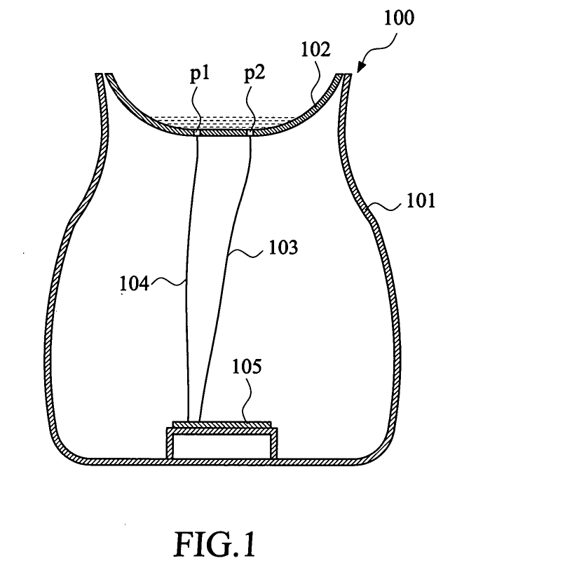

FIG. 1 shows a schematic side view of the present invention of an essential oil burner with an automatic power-off control circuit. An essential oil burner 100 includes a main body 101 and an essential oil bowl 102 for placing essential oil of an appropriate quantity. The essential oil bowl 102 includes a pair of detection electrodes p1, p2 at appropriate locations for detecting the presence of essential oil in essential oil bowl 102. The signal indicating the presence of the essential oil detected by detection electrodes p1, p2 is transmitted through a pair of signal lines 103, 104 to a circuit board 105, which is appropriately located inside main body 101 of essential oil burner 100.

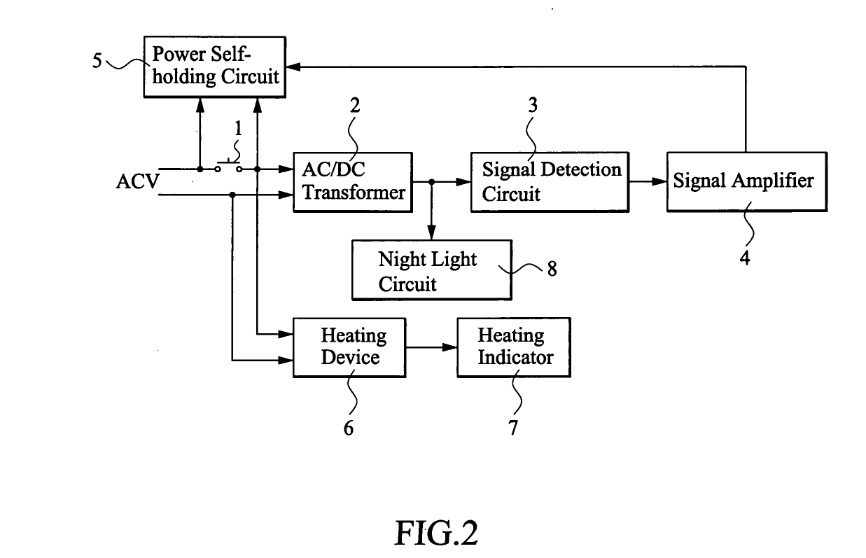

FIG. 2 shows a functional block diagram of the control circuit of the present invention. The block diagram includes a power-on switch 1, an AC/DC transformer 2, a signal detection circuit 3, a signal amplifier 4, a power self-holding circuit 5, a heating device 6, a heating indicator 7, and a night light circuit 8.

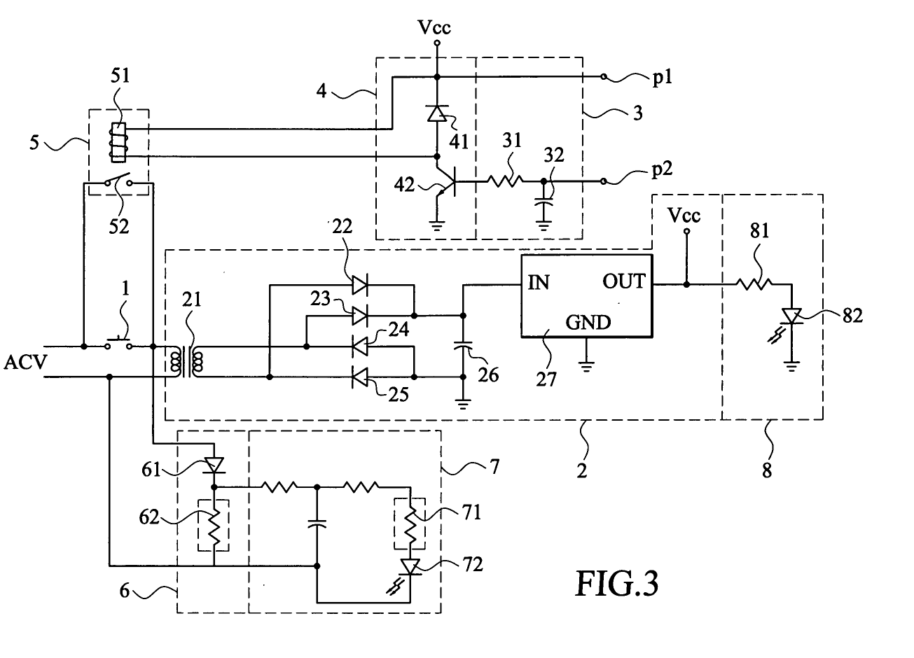

FIG. 3 shows a detailed control circuit of the present invention. The power-on switch 1 is connected between an AC power source ACV and the AC/DC transformer 2. The AC/DC transformer 2 can transform the AC current from AC power source ACV into a DC current with an appropriate voltage Vcc. The AC/DC transformer 2 includes a transformer 21, four diodes 22, 23, 24, 25 forming a bridge-type full-wave rectifier, a capacitor 26, and a power regulator 27.

the signal detection circuit 3 is connected to the detection electrodes p1, p2, which are located appropriately on the essential oil bowl 102 of the essential oil burner 100. The signal detection circuit 3 includes a resistor 31 and a capacitor 32. The signal generated by the signal detection circuit 3 is amplified by the signal amplifier 4. The signal amplifier 4 includes a diode 41 and a transistor 42 as the amplification device.

Both ends of the power-on switch 1 are connected, in parallel, to the power self-holding circuit 5. When the power-on switch 1 is pressed and the signal detection circuit 3 detects the presence of essential oil in the bowl 102, the power self-holding circuit 5 allows AC power source ACV to supply the power to the heating device 6 in order to heat the essential oil in the bowl 102. When the signal detection circuit 3 detects that the essential oil in the bowl 102 is used up, the power self-holding circuit 5 relieves its self-holding state and cuts off the power supply to the heating device 6.

The power self-holding circuit 5 includes a relay whose coil 51 is connected to the output end of the signal detection circuit 3, and the both ends of the normally open (N/O) contact 52 is connected in parallel with both ends of the power-on switch 1. When the power-on switch 1 is pressed and signal detection circuit 3 detects the presence of essential oil in the bowl 102, a control signal is generated to excite the coil 51 of the relay, and N/O contact 52 is closed.

The present invention of essential oil burner with an automatic power-off control circuit further includes a night light circuit 7, including a light sensitive resistor 71, an LED indicator 72, and relevant electronic elements. The light sensitive resistor 71 and the indicator 72 are connected in series and the connected in parallel to the heating device 6. When the light reaches a predefined lower level (as in the evening), the resistance of the light sensitive resistor 71 changes and the LED indicator 72 lights up.

The present invention of essential oil burner with an automatic power-off control circuit further includes a heating indicator 8, including a resistor 81 connected in series with an LED indicator 82. The heating indicator 8 is connected to DC voltage Vcc generated by the AC/DC transformer 2, so that it indicates the heating status when the power self-holding circuit 5 is in the self-holding state and power is supplied to the heating device 6.

From the aforementioned embodiment, it is obvious that the present invention of an essential oil burner with an automatic power-off control circuit allows the essential oil burner to automatically power off when it detects that the essential oil in the bowl is used up. It improves the safety of the essential oil burner. Further more, the self-holding feature of the present invention allows the user to only press the power-on switch to activate the burner, and the control circuit will continue the power supply to the heating device with a self-holding feature. Hence, it improves the operational convenience and provides highly usable value to the industry.

While the invention has been described in connection with what is presently considered to the most practical and preferred embodiment, it is to be understood that the invention is not to be limited to the disclosed embodiment, but on the contrary, is intended to cover various modifications and equivalent arrangement included within the spirit and scope of the appended claims.

1. An automatic power-off control circuit of an essential oil burner for controlling an AC power supply to the essential oil burner, the essential oil burner comprising a bowl for placing essential oil, the control circuit comprising:
an AC/DC transformer for transforming the AC power supply to a DC voltage;
a power-on switch connected in series between the AC power supply and the AC/DC transformer;
a heating device connected to the AC power supply, and connected between the power-on switch and the AC/DC transformer, for heating the essential oil in the bowl when the power-on switch being closed;
a signal detection circuit having two detecting electrodes located on the bowl of the essential oil burner for detecting the essential oil in the bowl;
a signal amplifier for receiving and amplifying a control signal from the signal detection circuit; and
a self-holding circuit having a normally open contact, the self-holding circuit connected in parallel with the power-on switch, the normally open contact being controlled by a control signal from an output end of the signal amplifier;
where when the power-on switch is pressed and the signal detection circuit detecting the essential oil in the bowl, the AC power supply is supplied to the heating device through the power-on switch to heat the essential oil in the bowl and the power-on switch staying in a closed state when the normally open contact of the self-holding circuit is closed, while the signal detecting circuit detecting the essential oil in the bowl being used up, the normally open contact of the self-holding circuit becoming open, causing the power-on switch becoming open, and cutting off the AC power supply to the heating device.

2. The control circuit as claimed in claim 1, wherein the self-holding circuit comprises a relay with a coil connected to the signal detection circuit, and with an normally open contact having both its ends connected in parallel with the power-on switch; when the power-on switch being pressed and the signal detection circuit detecting the essential oil in the bowl, a control signal is issued to excite the coil of the relay and close the normally open contact.

3. The control circuit as claimed in claim 1, further comprising a heating indicator connected to an output end of the AC/DC transformer to indicate that the normally open contact of the self-holding circuit is closed and the AC power supply is supplied to the heating device.

4. The control circuit as claimed in claim 1, further comprising a night light circuit having a light sensitive resistor and an indicator, the light sensitive resistor and the indicator being connected in series and connected to the heating device.
